(19) United States
(12) Patent Application Publication
(10) Pub. No.: US 2005/0234548 A1
(43) Pub. Date: Oct. 20, 2005

(54) Artifical auris interna

(76) Inventor: Mitsuhiro Yuasa, Tokyo (JP)

Correspondence Address: CROWELL & MORING LLP;INTELLECTUAL PROPERTY GROUP, P.O. BOX 14300, WASHINGTON, DC, 20044-4300, US

(21) Appl. No.: 10/525,307

(22) PCT Filed: Aug. 21, 2003
(86) PCT No.: PCT/JP03/10597

§ 371 Date: Feb. 22, 2005

(30) Foreign Application Priority Data
Aug. 23, 2002 (JP) 2002-243426

(51) Int. Cl. A61F 2/18

(52) U.S. Cl. 623/10; 607/57

(57) ABSTRACT

A fishbone sensor (21) has a plurality of resonators resonating with sounds having different frequencies from each other and converts the vibration of each resonator into a signal corresponding to each vibration level. An amplifying circuit (22) amplifies the signal converted by the fishbone sensor (21) by a predetermined gain and supplies it to an external switch circuit (23). The external switch circuit (23) switches signal supply paths and sequentially sends supplied signals via an external antenna (24). An internal switch circuit (32) switches signal supply paths and sequentially supplies the signals sent via an internal antenna (31) to a plurality of electrodes (4a), thereby stimulating the nerves in the cochlea.

### TECHNICAL FIELD

The present invention relates to an artificial ear.

### BACKGROUND ART

Man can recognize a sound when a nerve in the cochlea, a part of the internal ear, is stimulated.

Conventional artificial ears for helping the sense of hearing of the hearing-impaired people have a plurality of electrodes which are connected to the nerves in the cochlea, and directly stimulate nerves corresponding to the frequencies of sounds occurring in the surroundings with electricity.

The sounds occurring in the surroundings are collected by a microphone and divided into respective frequencies through signal processing by a DSP (Digital Signal Processor). Each sound having its own frequency is sent in the form of an electric signal to an electrode connected to a nerve corresponding to the frequency.

However, real-time sound processing by a DSP has a problem that it cannot achieve both a low level of power consumption and a high resolution simultaneously.

For example, in order to process a sound in real time at a low level of power consumption, it is necessary to reduce the number of frequencies to be processed, i.e., the number of electrodes for stimulating the nerves. However, this makes it impossible to realize a high resolution, and the sound to be perceived is unclear.

In order to process a sound to be perceived clearly and in real time, it is necessary to increase the number of frequencies to be processed, i.e., the number of electrodes for stimulating the nerves. This enormously increases the processes of the DSP and a low level of power consumption cannot be achieved.

For the reasons described above, the number of electrodes remains around 10 to 25 in the conventional artificial ears.

### DISCLOSURE OF INVENTION

Accordingly, an object of the present invention is to provide an artificial ear that realizes both a low level of power consumption and a high resolution simultaneously.

To achieve the above object, an artificial ear according to the present invention comprises: a sending unit (2) configured to convert a sound having a predetermined frequency into an electric signal and send the electric signal; and a reception unit (3) configured to receive the sent electric signal and apply it to a predetermined nerve in a cochlea, wherein

the sending unit (2) includes:
a plurality of resonators (21b) which have resonant frequencies different from each other and vibrate with sounds having same frequencies as the resonant frequencies;
a conversion section (21) configured to convert vibration of each of the plurality of resonators (21b) into a signal corresponding to level of the vibration; and
a sending section (28) configured to send a predetermined signal among signals converted by the conversion section (21) to the reception unit (3), and the reception unit (3) includes:
a plurality of electrodes (4a) which are connected to nerves present in the cochlea and each corresponding to different frequencies from each other; and
a supply section (34) configured to supply a signal supplied from the sending section (28) to a predetermined electrode among the plurality of electrodes (4a) thereby stimulating a nerve corresponding to a predetermined frequency.

In the above-described configuration, the sending unit (2) may further include an amplifying section (22) configured to amplify a signal converted by the conversion section (21) by a gain which varies in accordance with the respective resonant frequencies possessed by the plurality of resonators (21b).

In the above-described configuration, the sending section (28) may include a first selection section (23) configured to select a signal to be sent to the reception unit (3) from signals amplified by the amplifying section (22).

In the above-described configuration, the supply section (34) may include a second selection section (32) configured to select an electrode (4a) to which a signal from the sending section (28) is to be supplied.

In the above-described configuration, the sending section (28) may send a start signal representing a start of operation by the first selection section (23) and an end signal representing an end of operation by the first selection section (23) to the reception unit (3) in order to synchronize selection operations of the first selection section (23) and second selection section (32) with each other, and

the second selection section (32) may start operating in response to the start signal and finishes operating in response to the end signal.

In the above-described configuration, the sending unit (2) may further include a storage section (25) configured to store gains for the respective resonant frequencies possessed by the plurality of resonators (21b).

### BRIEF DESCRIPTION OF DRAWINGS

FIG. 1 is a block diagram of an artificial ear according to an embodiment of the present invention;

FIG. 2 is a block diagram of a fishbone sensor possessed by a sound processing unit that constitutes the artificial ear of FIG. 1;

FIG. 3 is a flowchart showing a signal sending process performed by an external switch circuit of the sound processing unit that constitutes the artificial ear of FIG. 1;

FIG. 4 is a flowchart showing a signal reception process performed by an internal switch circuit of a reception unit that constitutes the artificial ear of FIG. 1; and

FIG. 5 is another block diagram of the artificial ear according to the embodiment of the present invention.

### BEST MODE FOR CARRYING OUT THE INVENTION

An artificial ear according to an embodiment of the present invention will be explained below with reference to the drawings.

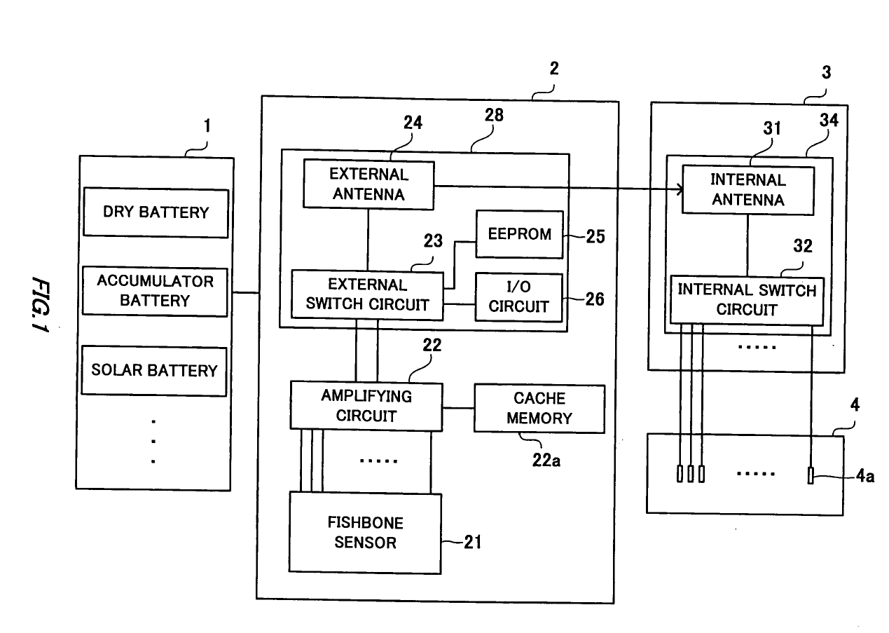

The artificial ear according to the embodiment of the present invention comprises a power source unit 1, a sound processing unit 2, a reception unit 3, and an electrode section 4, as shown in FIG. 1.

The power source unit 1 comprises at least one of a dry battery, an accumulator battery, a solar battery, a fuel battery, and a thermal power generator, etc. as shown in FIG. 1, and supplies power to the sound processing unit 2.

The sound processing unit 2 is set near the external ear by, for example, being caught on the auricle or in the earhole like an earphone. The sound processing unit 2 works by the power supplied from the power source unit 1, and converts a sound having a predetermined frequency, among sounds occurring in the surroundings, into an electric signal. The sound processing unit 2 sends the converted electric signal to the reception unit 3 in the form of a radio wave. The specific configuration of the sound processing unit 2 will be described later.

The reception unit 3 is implanted, for example, under the scalp near the external ear, and receives a radio wave from the sound processing unit 2. The reception unit 3 supplies the electric signal supplied in the form of a radio wave to the electrode section 4. The specific configuration of the reception unit 3 will be described later.

The electrode section 4 includes a plurality of electrodes 4a which are connected to the nerves in the cochlea, and stimulates the nerves in the cochlear by applying thereto the electric signal supplied from the reception unit 3. The plurality of electrodes 4a are respectively connected to nerves corresponding to the frequencies of sounds detected by the sound processing unit 2.

Next, the specific configuration of the sound processing unit 2 will be explained.

As shown in FIG. 1, the sound processing unit 2 comprises a fishbone sensor 21, an amplifying circuit 22, an external switch circuit 23, an external antenna 24, an EEPROM (Electrically Erasable Programmable Read Only Memory) 25, and an I/O (Input/Output) circuit 26.

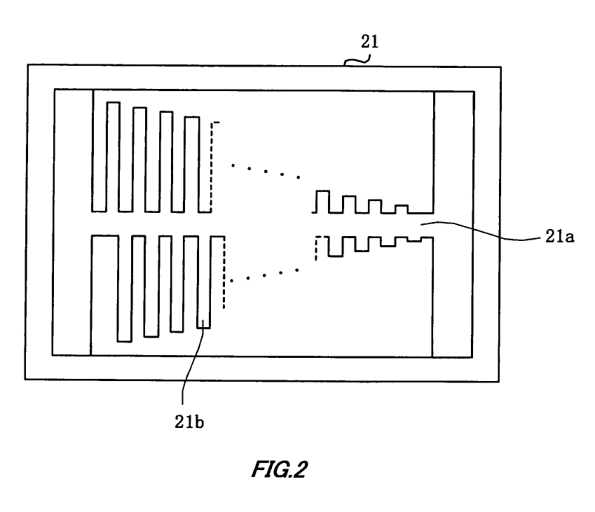

As shown in FIG. 2, the fishbone sensor 21 has a support shaft 21a and a plurality of cantilevers (resonators) 21b. The plurality of cantilevers 21b are formed on both sides of the support shaft 21a and have their one end fixed on the support shaft 21a.

The plurality of cantilevers 21b have their own different resonant frequencies. The material and shape of each cantilever 21b are selected such that these resonant frequencies can be uniformly distributed in the man's audio range. The cantilevers 21b are formed in a number corresponding to a frequency at which a sound occurring in the surroundings can be clearly perceived by man (for example, the number being 254).

When a sound occurring in the surroundings propagates through the support shaft 21a, the cantilever 21b that corresponds to the frequency contained in the propagating sound vibrates at a strength corresponding to the strength of the sound having the corresponding frequency.

The fishbone sensor 21 further has a detection circuit (unillustrated) which converts the vibration of each cantilever 21g into an electric signal. The vibration of each cantilever 21b is detected by the detection circuit and converted into a signal having a level corresponding to the strength of the vibration.

The detection circuit is, for example, a capacitor having the cantilever 21b functioning as one electrode thereof, and can detect the vibration of the cantilever 21b as changes in the capacitance of the capacitor.

After a sound is collected by a microphone, an output therefrom may be connected to a piezoelectric element provided in the fishbone sensor 21. In this case, the size of the unit to be set around the external ear can be reduced.

The fishbone sensor 21 outputs a signal generated in the way described above and having a level corresponding to the vibration level of each cantilever 21b to the amplifying circuit 22.

The amplifying circuit 22 connects a signal supply path between itself and the external switch circuit 23 in accordance with the control of the external switch circuit 23, amplifies a signal supplied from the fishbone sensor 21 by a predetermined gain, and outputs the amplified signal to the external switch circuit 23.

The amplifying circuit 22 has a cache memory 22a for accumulating gains to be described later which are stored in the EEPROM 25, and amplifies the signal from the fishbone sensor 21 by the gain accumulated in the cache memory 22a.

The amplifying circuit 22 further has a timer (unillustrated) for counting the time in which a signal supply path is connected. The timer start counting a preset connection time when a signal supply path is connected between the amplifying circuit 22 and the external switch circuit 23. When the predetermined connection time passes, the amplifying circuit 22 disconnects the signal supply path that leads to the external switch circuit 23.

The external switch circuit 23 controls the amplifying circuit 22 to sequentially switch signal supply paths between itself and the amplifying circuit 22 at predetermined timings. In other words, the external switch circuit 23 sequentially selects signals to be sent, from among signals amplified by the amplifying circuit 22 at predetermined timings one signal by one, and sends the signal to the reception unit 3 via the external antenna 24.

The EEPROM 25 stores the gain for each frequency, that is used by the amplifying circuit 22 for amplifying a signal. The strength of an electric signal stimulating a nerve in the cochlea varies depending on individual persons and frequencies. Hence, the gain used by the amplifying circuit 22 is set for each frequency suitably for the user of the artificial ear.

The I/O circuit 26 is used for rewriting the gains stored in the EEPROM 25.

As described above, the external switch circuit 23, the external antenna 24, the EEPROM 25, and the I/O circuit 26 constitute a sending section 28 for sending a predetermined signal, among signals converted by a conversion section, to the reception unit 3.

Next, the specific configuration of the reception unit 3 will be explained.

As shown in FIG. 1, the reception unit 3 comprises an internal antenna 31 and an internal switch circuit 32.

The internal antenna 31 receives a signal sent in the form of a radio wave from the external antenna 24 via the scalp, and supplies it to the internal switch circuit 32.

The internal switch circuit 32 works by electricity supplied in the form of an electromagnetic wave via the internal antenna 31, and sequentially switches signal supply paths between the internal antenna 31 and the plurality of electrodes 4a at predetermined timings. In other words, the internal switch circuit 32 sequentially selects electrodes 4a to which a signal is to be supplied at predetermined timings one electrode by one, and distributes signals supplied from the internal antenna 31 to the plurality of electrodes 4a.

In this way, the internal antenna 31 and the internal switch circuit 32 constitute a supply section 34 for supplying a signal supplied from the sending section 28 to a predetermined electrode among the plurality of electrodes, thereby stimulating a nerve corresponding to a predetermined frequency.

The external switch circuit 23 and the internal switch circuit 32 are designed in advance in a manner that the timings at which signal supply paths are switched are synchronous between them. Further, the connection time counted by the timer of the amplifying circuit 22 is preset so as to correspond to the interval at which the external switch circuit 23 and the internal switch circuit 32 switch signal supply paths.

Next, the operation of the artificial ear according to the embodiment of the present invention will be explained.

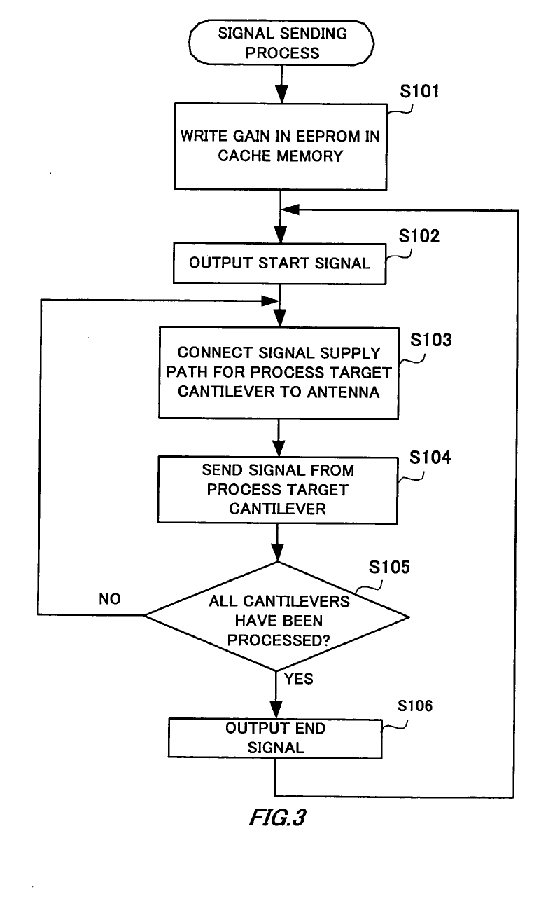

When the sound processing unit 2 is turned on, the external switch circuit 23 starts a signal sending process shown in FIG. 3.

First, the external switch circuit 23 reads the gain for each frequency from the EEPROM 25 and writes it in the cache memory 22a possessed by the amplifying circuit 22 (step S101). As a result, the amplifying circuit 22 becomes able to amplify signals corresponding to respective frequencies supplied from the fishbone sensor 21 by predetermined gains.

When a sound occurs in the surroundings, the occurring sound propagates through the support shaft 21a of the fishbone sensor 21. Due to this, the cantilever 21b that corresponds to the frequency contained in the propagating sound vibrates at a strength corresponding to the strength of the sound having the corresponding frequency.

The vibration of each cantilever 21b is converted by the unillustrated detection circuit into a signal having a level corresponding to the strength of the vibration, and supplied to the amplifying circuit 22 to be amplified.

After writing the gain for each frequency in the cache memory 22a, the external switch circuit 23 outputs a start signal representing the start of an operation for switching signal supply paths to the amplifying circuit 22, and also sends it to the reception unit 3 via the external antenna 24 (step S102).

Because of the start signal, the timing at which the external switch circuit 23 starts switching operation and the timing at which the internal switch circuit 23 starts switching operation can securely be synchronized.

The amplifying circuit 22 resets the timer in response to the start signal from the external switch circuit 23.

After outputting the start signal, the external switch circuit 23 controls the amplifying circuit 22 and switches signal supply paths to connect a signal supply path for the process target cantilever 21b to the externalantenna 24 (step S103).

Specifically, the external switch circuit 23 outputs a switching signal instructing switching of signal supply paths to the amplifying circuit 22. In response to the switching signal from the external switching circuit 23, the amplifying circuit 22 connects a signal supply path for the process target cantilever 21b to the external switch circuit 23. As a result, the process target cantilever 21b and the external antenna 24 are connected to each other.

When the signal sending process is started, a cantilever 21b corresponding to a preset frequency (for example, the highest frequency) is selected as the process target cantilever 21b.

The signal from the process target cantilever 21b is amplified by the amplifying circuit 22 by the predetermined gain stored in the cache memory 22a and supplied to the external switch circuit 23.

The external switch circuit 23 sends the signal from the process target cantilever 21b supplied from the amplifying circuit 22 to the reception unit 3 via the external antenna 24 (step S104).

As described above, the timer possessed by the amplifying circuit 22 starts counting the preset connection time in response to the connection of a signal supply path. When the predetermined connection time passes, the amplifying circuit 22 automatically disconnects the signal supply path between itself and the external switch circuit 23.

When the signal supply path is disconnected, the external switch circuit 23 determines whether the above-described process has been performed for all the cantilevers 21b (or for all the frequencies) (step S105).

In a case where determining that the process has not been performed for all the cantilevers 21b (or for all the frequencies) (step S105; NO), the external switch circuit 23 returns to the above step S103 to perform the above-described process for the next cantilever 21b (or frequency).

To the contrary, in a case where determining that the process has been performed for all the cantilevers 21b (or for all the frequencies) (step S105; YES), the external switch circuit 23 outputs an end signal representing the end of the operation for switching the signal supply paths to the amplifying circuit 22 and also sends it to the reception unit 3 via the external antenna 24 (step S106).

Because of the end signal, the timing at which the external switch circuit 23 ends the switching operation and the timing at which the internal switch circuit 32 ends the switching operation can securely be synchronized.

In the way described above, the process for a sound occurring at an instant is finished. In the sound processing unit 2, the process from the above-described steps S102 through S106 is repeated while the power is turned on, thereby sounds occurring one after another are processed and sent to the reception unit 3.

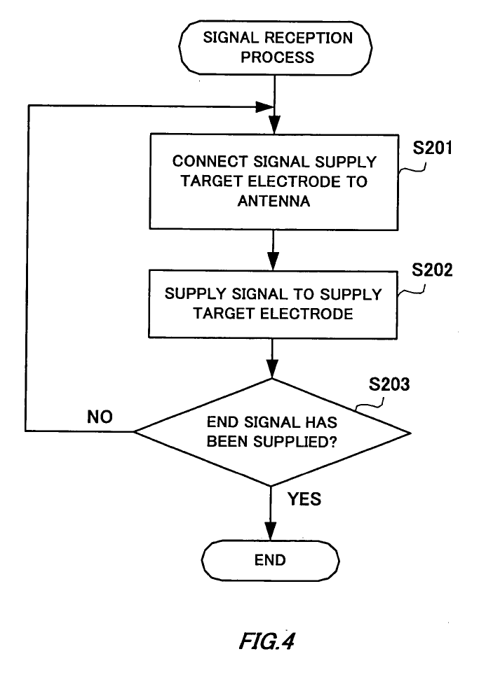

In the meantime, the internal switch circuit 32 in the reception unit 3 starts operating in response to the start signal supplied from the sound processing unit 2 via the internal antenna 31, and starts a signal reception process shown in FIG. 4.

First, the internal switch circuit 32 switches signal supply paths in the manner of time division so as to synchronize with the sound processing unit 2, and connects an electrode 4a which is connected to a nerve corresponding to the frequency of the process target cantilever 21b to the internal antenna 31 (step S201). By doing so, the internal switch circuit 32 selects the electrode 4a connected to the nerve corresponding to the frequency of the process target cantilever 21b as the signal supply target.

When the signal reception process is started, the internal switch circuit 32 selects an electrode 4a connected to a nerve corresponding to a preset frequency (for example, the highest frequency) as the signal supply target.

The internal switch circuit 32 supplies the signal supplied via the internal antenna 31 to the selected supply target electrode 4a (step S202).

The nerve to which the supply target electrode 4a is connected is stimulated by the supplied signal. Due to this, the user of the artificial ear can perceive the sound having the frequency corresponding to the stimulated nerve.

After supplying the signal, the internal switch circuit 32 determines whether or not an end signal has been supplied from the sound processing unit 2 (step S203).

In a case where determining that an end signal has not been supplied (step S203; NO), the internal switch circuit 32 returns to the above-described step S201 and performs the above-described process for the next electrode 4a.

To the contrary, in a case where determining that an end signal has been supplied (step S203; YES), the internal switch circuit 32 finishes the signal sending process and stops operating.

In the way described above, a sound occurring at a given instant is processed and transmitted to the nerve of the user.

As described above, using the fishbone sensor 21 having the cantilevers 21b that resonate with a variety of frequencies eliminates the need of performing complicated signal processing performed by the conventional DSPs. It is therefore possible to largely increase the number of frequencies to be processed as compared with those conventionally processed, while at the same time suppressing increase in the power consumed. As a result, a clearer and finer sound than obtained in a conventional manner can be perceived at a low level of power consumption.

Further, as described above, the internal switch circuit 32 starts operating in response to a start signal supplied from the sound processing unit 2 and stops operating in response to an end signal. Therefore, the supply path switching operations performed by the external switch circuit 23 and internal switch circuit 32 can be more securely synchronized.

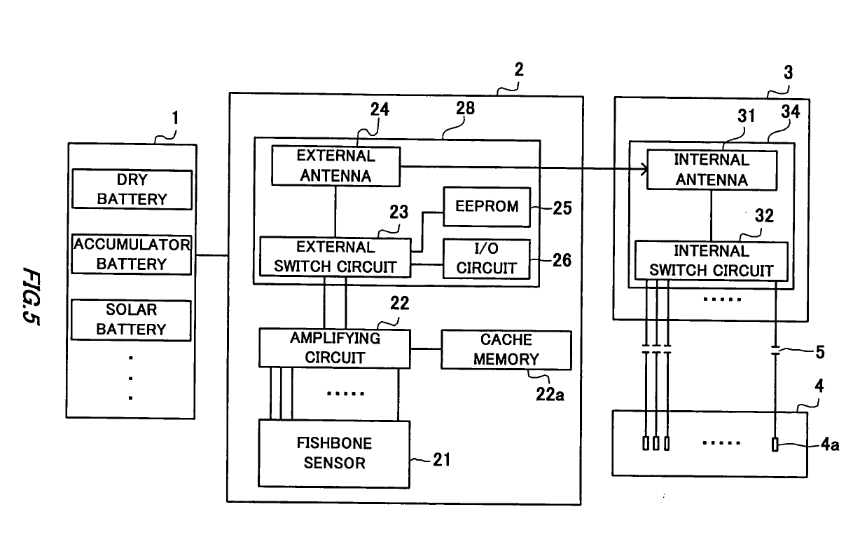

The above-described artificial ear may be provided with a capacitor 5 between the internal switch circuit 32 and the electrodes 4a as shown in FIG. 5, in order to smooth pulse signals supplied to the electrodes 4a in the time division manner from the internal switch circuit 32a.

To make conversational sounds much clearer, the density of cantilevers 21b for the human vocalization frequency band may be increased with respect to the densities for other frequency bands.

The fishbone sensor 21, the amplifying circuit 22, the external switch circuit 23, the EPROM 25, and the I/O circuit 26 may be formed on one chip by a micromachine technology, a semiconductor manufacturing technique, etc. This makes it possible to realize a compact sound processing unit 2.

Cantilevers 21b which resonate with sounds having frequencies out of the man's audio range may be provided so that sounds other than those in the audio range can be transmitted to the nerves in the cochlear in the form of electric signals. With this configuration, sounds varying in a broader range than that of ordinary people or dogs can be perceived, allowing application to special purposes such as military use, etc.

The present invention is based on Japanese Patent Application No. 2002-243426 filed on Aug. 23, 2002 and including specification, claims, drawings and summary. The disclosure of the above Japanese Patent Application is incorporated herein by reference in its entirety.

### Industrial Applicability

The present invention is very useful in industries concerning supports for the reduced hearing ability of hearing-impaired people or elderly people.

1. An artificial ear comprising: a sending unit (2) configured to convert a sound having a predetermined frequency into an electric signal and send the electric signal; and a reception unit (3) configured to receive the sent electric signal and apply it to a predetermined nerve in a cochlea, characterized in that
said sending unit (2) includes:
a plurality of resonators (21b) which have resonant frequencies different from each other and vibrate with sounds having same frequencies as the resonant frequencies;
a conversion section (21) configured to convert vibration of each of said plurality of resonators (21b) into a signal corresponding to level of the vibration; and
a sending section (28) configured to send a predetermined signal among signals converted by said conversion section (21) to said reception unit (3), and
said reception unit (3) includes:
a plurality of electrodes (4a) which are connected to nerves present in the cochlea and each corresponding to different frequencies from each other; and
a supply section (34) configured to supply a signal supplied from said sending section (28) to a predetermined electrode among said plurality of electrodes (4a) thereby stimulating a nerve corresponding to a predetermined frequency, and
said plurality of resonators (21b) have their ends at one side held independent from each other.

2. The artificial ear according to claim 1, characterized in that said sending unit (2) further includes an amplifying section (22) configured to amplify a signal converted by said conversion section (21) by a gain which varies in accordance with the respective resonant frequencies possessed by said plurality of resonators (21b).

3. The artificial ear according to claim 2, characterized in that said sending section (28) includes a first selection section (23) configured to select a signal to be sent to said reception unit (3) from signals amplified by said amplifying section (22).

4. The artificial ear according to claim 3, characterized in that said supply section (34) includes a second selection section (32) configured to select an electrode (4a) to which a signal from said sending section (28) is to be supplied.

5. The artificial ear according to claim 4, characterized in that said sending section (28) sends a start signal representing a start of operation by said first selection section (23) and an end signal representing an end of operation by said first selection section (23) to said reception unit (3) in order to synchronize selection operations of said first selection section (23) and second selection section (32) with each other, and
said second selection section (32) starts operating in response to the start signal and finishes operating in response to the end signal.

6. The artificial ear according to claim 2, characterized in that said sending unit (2) further includes a storage section (25) configured to store gains for the respective resonant frequencies possessed by said plurality of resonators (21b).

7. The artificial ear according to claim 1, characterized in that said plurality of resonators (21b) have their ends at the other side connected to a support shaft (21a) and supported by said support shaft (21a).

8. The artificial ear according to claim 7, characterized in that said plurality of resonators (21b) are arranged on both sides of said support shaft (21a).

9. The artificial ear according to claim 8, characterized in that said plurality of resonators (21b) have different lengths from each other and are arranged on said support shaft (21a) in an order of a larger length to a smaller length from one end toward the other end of said support shaft (21a).
